(19) United States
(12) Patent Application Publication
(10) Pub. No.: US 2026/0351390 A1
(43) Pub. Date: Nov. 5, 2026

(54) Perioperative Cobotic System for Integrated Surgical Assistance and Workflow Optimization

(71) Applicant: Peritas AI Inc., Fort Lauderdale, FL (US)

(72) Inventor: Martin Roche, Fort Lauderdale, FL (US)

(73) Assignee: Peritas AI Inc., Fort Lauderdale, FL (US)

(21) Appl. No.: 19/432,111

(22) Filed: Dec. 24, 2025

Related U.S. Application Data
(63) Continuation-in-part of application No. 19/195,731, filed on Apr. 30, 2025.

(51) Int. Cl. G16H 40/20; B25J 9/16; G16H 10/60

(52) U.S. Cl. CPC G16H 40/20; B25J 9/1661; G16H 10/60

(57) ABSTRACT

An adaptive perioperative cobotic system is disclosed for optimizing surgical workflow through real-time monitoring, contextual task allocation, and predictive coordination. The system includes a collaborative robot equipped with articulated arms and multimodal sensors for performing perioperative support tasks. A workflow recognition module analyzes optical, audio, and motion-tracking data to classify procedural phases and surgical team activity. Based on the recognized workflow state, an adaptive task allocation engine dynamically assigns cobotic actions. A predictive workflow optimization engine leverages historical case data and real-time inputs to anticipate upcoming procedural steps and optimize resource deployment. An integrated tool and asset management module tracks instrument usage and tray composition and generates alerts for deviations or readiness thresholds. The system interfaces with electronic health record systems to retrieve procedural plans and log intraoperative events. Safety mechanisms enforce sterile-field compliance and enable manual override.

### CROSS-REFERENCE TO RELATED APPLICATIONS

This patent application is a continuation-in-part of US Patent Application Ser. No. 19/195,731 filed on Apr. 30, 2025, which claims the benefit of priority to U.S. Provisional Patent Application No. 63/640,524, filed on Apr. 30, 2024, the entirety of which is incorporated herein by reference.

### TECHNICAL FIELD

The present invention relates generally to robotic systems for surgical environments and, more particularly, to adaptive perioperative cobotic systems configured for surgical workflow monitoring, task automation, and predictive procedural optimization.

### BACKGROUND

Modern surgical procedures are increasingly complex, involving multi-disciplinary teams, diverse instruments, and highly dynamic intraoperative workflows. Successful outcomes depend not only on clinical skill but also on precise coordination, timely access to tools, and continuous situational awareness. Despite advances in imaging and navigation, the operating room (OR) remains a high-pressure, variable environment where inefficiencies, delays, or procedural deviations can compromise outcomes.

Collaborative robots, or “cobots,” have emerged as promising tools in perioperative environments, offering assistive capabilities such as instrument delivery, imaging positioning, or environmental adjustment. However, existing cobotic systems typically operate in predefined modes and lack contextual awareness of the surgical workflow. These systems are reactive rather than adaptive, with limited capacity to respond dynamically to real-time changes such as unexpected delays, complications, or team-member substitutions.

Furthermore, conventional OR automation solutions are siloed and fragmented, often addressing narrow tasks, such as tray tracking or inventory management, without integrating broader workflow intelligence. While some systems capture data or recognize procedural steps, they generally do not synthesize multimodal information (e.g., visual, auditory, motion tracking) into actionable workflow insights. Nor do they employ learning-based systems to improve surgical coordination over time.

A particular gap exists in systems capable of recognizing procedural phases, interpreting team behavior, predicting upcoming surgical steps, and allocating cobot resources accordingly. Without such capabilities, cobots remain underutilized, lacking the adaptability to provide meaningful support across different surgical specialties and procedural variations.

There is thus a need for a perioperative cobotic system that integrates advanced sensing, machine learning-based workflow recognition, adaptive task allocation, and predictive modeling. Such a system should interface with electronic health records (EHRs) to guide preoperative planning and intraoperative execution, while preserving sterile-field compliance and enabling real-time human-cobot collaboration. An adaptive system of this kind would improve efficiency, reduce cognitive load on surgical staff, and enhance both safety and procedural consistency in the OR.

### SUMMARY

The present invention provides an adaptive perioperative cobotic system configured to optimize surgical workflow through real-time monitoring, contextual task allocation, and predictive support. The system includes a collaborative robot (cobot) equipped with articulated arms and surgical-environment sensors for performing a range of perioperative tasks, including instrument delivery, environmental adjustments, and intraoperative coordination. The cobot is capable of autonomous or semi-autonomous operation and can navigate dynamically within the operating room while complying with sterile-field boundaries and team movement constraints.

A workflow monitoring and recognition system uses multimodal sensor inputs, including optical, audio, and motion tracking, to observe surgical team behavior, instrument usage, and procedural events. Machine learning algorithms classify discrete workflow phases, such as incision, retraction, and closure, and detect intraoperative task transitions and anomalies.

Based on the recognized workflow state, an adaptive task allocation engine dynamically assigns cobotic actions and reprioritizes tasks in response to changing conditions, such as unexpected delays or changes in team composition. The system further includes an integrated tool and asset management module to track instrument usage in real time, prevent duplication, and alert users when reprocessing or replenishment thresholds are triggered.

A predictive workflow optimization engine analyzes historical case data, surgeon-specific preferences, and procedure type to anticipate upcoming workflow events. This engine supports pre-positioning of tools, staff coordination, and timing predictions, while continuously learning from completed procedures to refine future predictions and task sequencing.

A context-aware user interaction system provides OR staff with real-time updates, procedural cues, and adaptive control via voice commands, touchscreen displays, and augmented reality interfaces. The system interfaces with hospital electronic health record (EHR) and scheduling platforms to retrieve patient-specific plans and to log intraoperative events and workflow deviations for postoperative documentation.

The cobotic system includes safety mechanisms that monitor environmental and procedural constraints, ensure sterile-zone compliance, and provide manual override and escalation protocols to support workflow integrity in dynamic surgical settings.

### BRIEF DESCRIPTION OF DRAWINGS

The foregoing and other objects, aspects, features, and advantages of the disclosure will become more apparent and better understood by referring to the following description taken in conjunction with the accompanying drawings, in which:

FIG. 1 is a perspective view of an adaptive perioperative cobot.

FIG. 2 is a block diagram of the system architecture.

FIG. 3 is a schematic of the workflow recognition system.

FIG. 4 is a diagram of the adaptive task allocation process.

FIG. 5 is a schematic of the integrated tool and asset management module.

FIG. 6 is a flowchart of the predictive workflow optimization engine.

FIG. 7 is a block diagram of user interface components.

FIG. 8 is a communication diagram showing integration between the cobotic system and hospital electronic health record and scheduling.

FIG. 9 is a safety compliance diagram.

These drawings are provided for illustrative purposes only and should not be construed as limiting the invention to the specific embodiments shown and described herein. Variations and modifications to these embodiments will be apparent to those skilled in the art, and all such variations and modifications are intended to be included within the scope of this disclosure.

### DETAILED DESCRIPTION

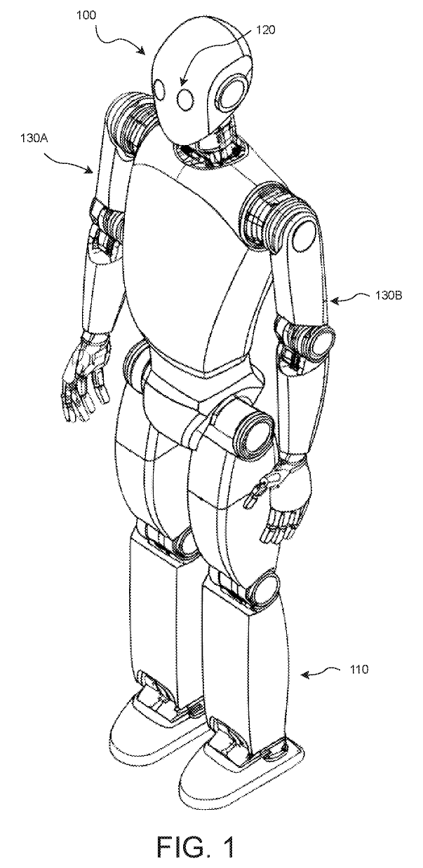

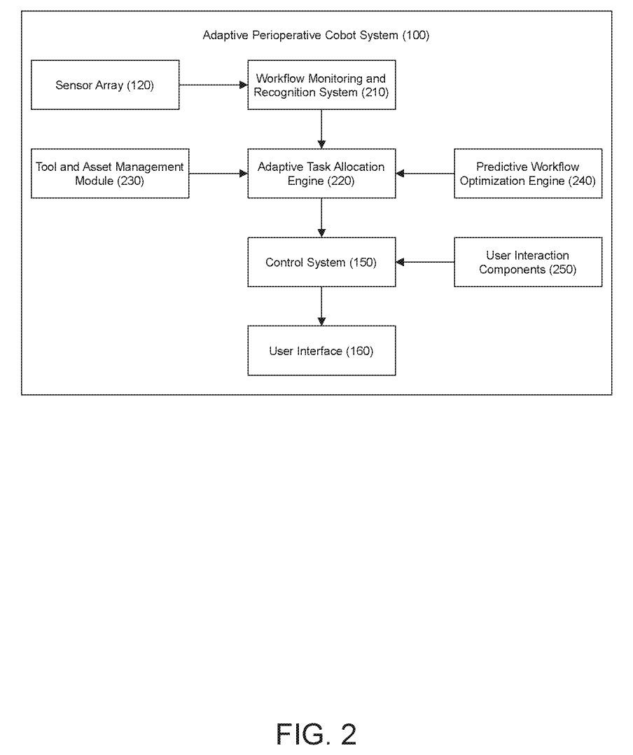

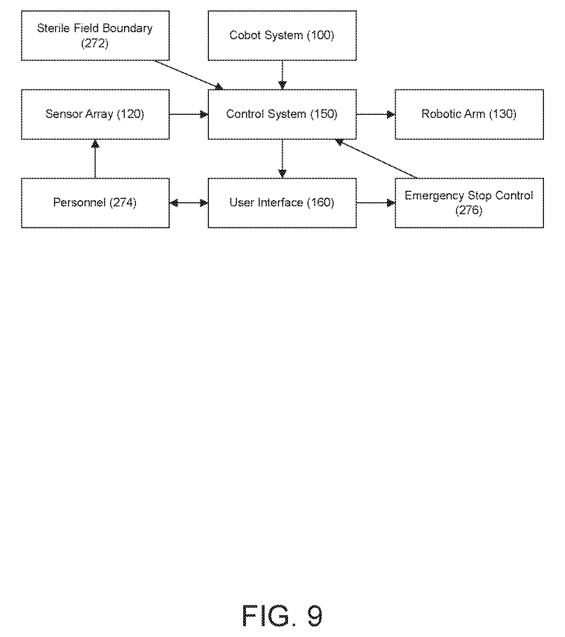

Referring now in detail to the drawings, FIG. 1 illustrates an adaptive perioperative cobot system 100. The cobot system 100 includes a stable base platform 110, one or more articulated robotic arms 130, and a multimodal sensor array 120. The base 110 serves as the foundation for the cobot, providing support and (if mobile) housing a drive mechanism for maneuverability within the OR. In some embodiments, the base 110 is a bipedal mobile platform. In other embodiments, the base 110 is a wheeled mobile platform equipped with motors and steering actuators to navigate the dynamic operating room environment. It may contain a power supply (e.g. batteries) and the main control system 150 (shown in FIG. 2, including onboard processors and memory) that govern the cobot’s operations. The articulated arms 130 (for example, arms 130A and 130B in a two-arm configuration) are mounted on the base 110 and are capable of multi-degree-of-freedom movement. These arms 130 enable the cobot to interact with surgical instruments, equipment, and its surroundings in a human-like manner. For instance, the arms 130 can fetch or hold instruments, adjust overhead lights or cameras, open cabinets, or assist in non-sterile tasks to support the surgical team. The cobot’s multimodal sensor array 120 is strategically distributed on the platform (e.g. on the arms and mast of the base) to provide 360-degree situational awareness. In the embodiment of FIG. 1, the sensor array 120 includes optical cameras, depth sensors (such as LiDAR), and audio sensors (microphones), among others. These sensors continuously capture information about the surgical scene, for example, visual data of the surgical field and instrument table, positions of personnel, ambient audio (surgeon’s voice commands or OR alarms), and even environmental parameters. By fusing inputs from these modalities, the cobot 100 can perceive and understand the surgical workflow in real time. The physical configuration shown in FIG. 1, with the base 110, arms 130, and sensor suite 120, is designed for collaborative integration into the perioperative workflow. The cobot 100 occupies roughly the footprint of a person or equipment cart and is designed with rounded edges and compliance in its joints to safely operate near humans. It is further equipped with safety sensors (as will be detailed with FIG. 9) that help it avoid collisions and respect sterile boundaries. In operation, the control system 150 (located within the base 110 in this embodiment) coordinates the base 110, arms 130, and sensors 120 so that the cobot can move about the OR without interfering with the surgical team or sterile field, yet be close enough to perform its assistance tasks.

Turning now to FIG. 2, a schematic block diagram of the system architecture is presented to illustrate the interconnectivity of the cobot’s functional components. The adaptive perioperative cobot system 100 is organized into several interconnected subsystems, all communicatively linked via the control system 150. As shown in FIG. 2, a workflow monitoring and recognition system 210 receives data streams from the sensor array 120 and analyzes them to determine the current phase of the surgical procedure. The workflow recognition system 210 may utilize computer vision and audio analysis, for example, processing the camera feed for visual cues (such as incision vs. closure activities) and the audio feed for verbal cues or device sounds, to classify the ongoing workflow phase (e.g., patient prep, incision, anatomic dissection, wound closure). The output of the recognition system 210 feeds into an adaptive task allocation engine 220, which dynamically decides how the cobot 100 should assist at any given time. The task allocation engine 220 uses the identified workflow phase and also monitors the team’s behavior and needs (for instance, detecting if a nurse’s hands are busy or if a surgeon is requesting an item) to assign or adjust tasks for the cobot. In parallel, the architecture includes an integrated tool and asset management module 230 that tracks surgical instruments and other assets in real-time. This module 230 interfaces with various sensors (such as RFID readers on the cobot or vision sensors from array 120 trained on the instrument tray) to monitor instrument usage and tray inventory. Information from the tool management module 230 is available to both the task allocation engine 220 and the workflow recognition system 210 (since instrument usage can be a cue to the procedure’s progress). The system further comprises a predictive workflow optimization engine 240 that operates in conjunction with a data store of historical cases and the live data coming from module 210. The predictive engine 240 analyzes trends, including surgeon-specific preferences, the current patient’s context, and past similar surgeries, to anticipate upcoming needs or potential bottlenecks. Its predictions inform the task allocation engine 220 (so the cobot can, for example, pre-position the next instrument or adjust its schedule) and can also be communicated to the surgical team as advance warnings or suggestions. Finally, the architecture integrates user interaction components 250, which encompass the various interfaces through which the surgical staff and the cobot communicate. The user interaction system 250 includes a local user interface 160 (e.g. a touchscreen display on the cobot or a tablet) for visual feedback and input, as well as voice recognition and speech output modules (tied into the cobot’s microphones and speakers) and an augmented reality interface. These components 250 allow the cobot to provide real-time cues to the team and to accept commands via spoken instructions, touch inputs, or hand gestures. All of the above subsystems 210, 220, 230, 240, and 250 are operatively coordinated by the central control system 150. The control system 150, which may be implemented as one or more onboard computers, manages data flow between modules, executes the decision algorithms, and ensures that the cobot’s physical actions (movement of base 110 or arms 130) are issued in a safe, timely manner according to the outputs of these subsystems. FIG. 2 thus provides a high-level overview of how sensor data, processing modules, and user interfaces interconnect within the cobot system 100 to adaptively optimize the surgical workflow.

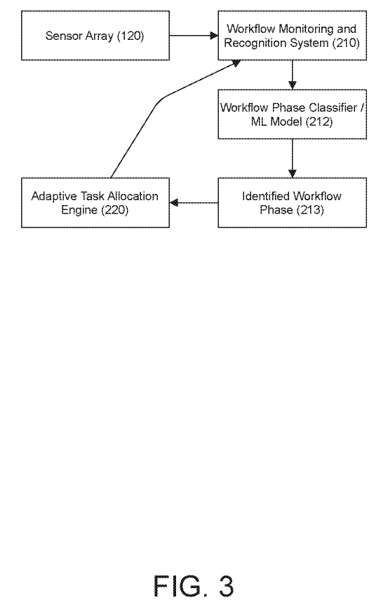

In FIG. 3, a schematic of the workflow recognition system 210 is shown in greater detail. As illustrated, multiple sensor input channels feed into a machine-learning-based workflow classification model 212. In this embodiment, the sensor array 120 provides several concurrent data streams: for example, a video feed 121 from an overhead camera observing the surgical field, an audio feed 123 from microphones capturing conversations and OR sounds, and motion or positional data 125 from trackers (which might include the cobot’s own joint sensors or external beacons tracking the movement of staff and equipment). These inputs are preprocessed (e.g., frames of video are analyzed for specific activities, audio is filtered for key phrases or tonal alerts) and then fed into the workflow phase classifier 212. The classifier 212 may comprise a trained machine learning model, such as a deep neural network or other pattern recognition algorithm, that has been trained on annotated surgical procedures to recognize distinct phases and important events. FIG. 3 diagrammatically shows the classifier 212 outputting an identified phase 213 (for instance, recognizing that the team has moved from the “pre-incision” phase to the “incision” phase based on visual cues like the surgeon holding a scalpel, or auditory cues like the surgeon announcing “begin incision”). The workflow monitoring system 210 can be configured to recognize standard surgical phases (e.g., anesthesia induction, incision, robotic utilization, main operation tasks, closing, dressing) as well as sub-tasks or transitions. In some embodiments, the recognition system 210 not only classifies the phase but also detects contextual indicators such as which surgical personnel are present and active, or whether an unexpected event (like a cautery alarm or a change in patient vital signs) has occurred. As shown in FIG. 3, the classifier’s real-time output 213 is communicated to other components (denoted by arrows to the task engine 220 and predictive engine 240), enabling the rest of the system to react appropriately. By continuously analyzing multimodal sensor data, the workflow recognition module 210 provides a live “situational awareness” of the procedure’s progress, which is fundamental for the cobot 100 to assist in an intelligent, phase-appropriate manner. In an embodiment, the workflow recognition system 210 improves over time through machine learning feedback: data from each surgery (including the phases detected and any errors or adjustments made) can be logged and used to retrain or refine the model 212, thereby increasing accuracy in future procedures.

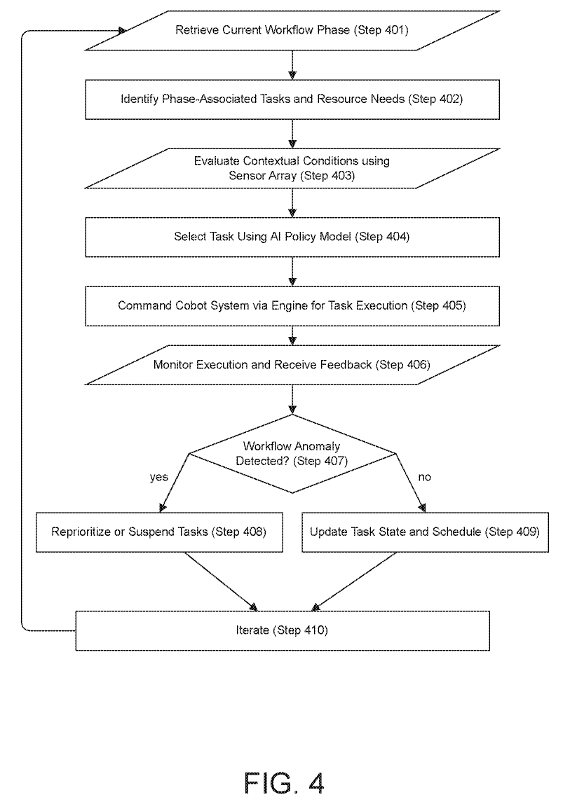

FIG. 4 illustrates an adaptive task allocation process executed by the adaptive task allocation engine 220. The process operates as a closed-loop control sequence driven by real-time workflow classification and contextual sensing within the operating room.

Retrieve Current Workflow Phase (Step 401). The adaptive task allocation engine 220 receives a currently identified workflow phase 213 from the workflow monitoring and recognition system 210, the workflow phase corresponding to a discrete stage of the surgical procedure.

Identify Phase-Associated Tasks and Resource Needs (Step 402). Based on the identified workflow phase 213, the engine 220 determines one or more candidate cobotic tasks and associated resource requirements by querying a procedural task model or task repository.

Evaluate Contextual Conditions (Step 403).The engine 220 evaluates real-time contextual data describing surgical team activity, personnel availability, workload state, and environmental conditions using sensor data acquired from the sensor array 120, including detection of occupied personnel or changes in team composition.

Select Task Using Policy Model (Step 404). The engine 220 applies a rules-based framework or an AI policy model 222 to map the identified workflow phase and evaluated contextual conditions to a specific cobotic action to be performed.

Command Cobotic Task Execution (Step 405). The engine 220 issues control commands to the cobot 100 to execute the selected task, including directing one or more articulated arms 130 to perform actions such as retrieving and presenting a surgical instrument 223 or preparing equipment associated with a closing phase.

Monitor Task Execution and Receive Feedback (Step 406). The engine 220 monitors execution of the assigned task and receives confirmation or sensory feedback 225 indicating task completion, execution status, or task interruption.

Detect Workflow Anomaly (Step 407). The engine 220 analyzes task execution feedback and procedural timing to determine whether a workflow anomaly 227 has occurred, including detection of unexpected delays, complications, or procedural interruptions.

Reprioritize or Suspend Tasks (Step 408). In response to detection of a workflow anomaly, the engine 220 reprioritizes pending tasks, suspends active cobot operations, or transitions the cobot 100 into a standby or support mode to avoid interference with surgical activities.

Update Task State and Schedule (Step 409). If no workflow anomaly is detected, or once the anomaly condition has resolved, the engine 220 updates a task queue or task state model and adjusts the cobot’s task schedule based on current procedural progress.

Iterative Closed-Loop Operation (Step 410). The adaptive task allocation process repeats continuously, enabling the engine 220 to dynamically modify, accelerate, decelerate, or reassign cobotic tasks in real time in response to changes in workflow phase, team behavior, or surgical tempo.

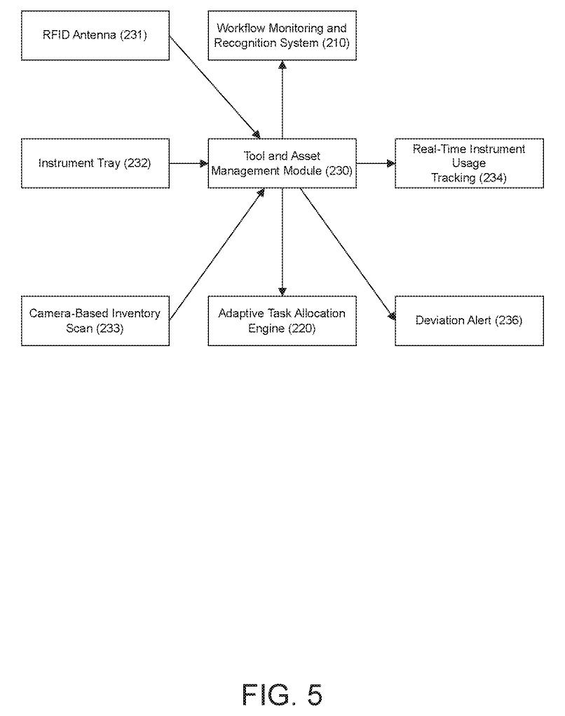

FIG. 5 provides a schematic illustration of the integrated tool and asset management module 230 in operation. In the example shown, the module 230 monitors the status and usage of surgical instruments and other assets (such as sponges, implants, or medications) in real time to ensure workflow efficiency and compliance with surgical plans. As depicted in FIG. 5, the cobot 100 is equipped with or connected to several sensing mechanisms for tracking instruments: one embodiment includes an RFID antenna 231 on the cobot or in the OR that detects RFID-tagged instruments entering or leaving the instrument tray 232, and another includes a camera-based inventory scan 233 that visually inspects the layout of instruments on a tray or table. The tool management module 230 aggregates these inputs to maintain a live inventory count and status report of all critical items. For instance, FIG. 5 shows the module 230 comparing the real-time instrument count against the expected inventory for the current procedure (which may be stored in a database or pulled from the EHR plan). If an instrument that should be present is missing or an extra instrument is introduced, the system flags a discrepancy. One feature illustrated in FIG. 5 is real-time instrument usage tracking 234: as surgery progresses, module 230 records each tool that the surgeon uses (detected either by vision, recognizing a specific tool in the surgeon’s hand, or by the tool being removed from/returned to the tray). This information is used to alert the team if, for example, a tool is not returned when expected (potentially indicating it’s misplaced or retained in the surgical site) or if a critical instrument has not been used when by this point it typically would (which could indicate a deviation from the standard procedure or a possible oversight). The module 230 also continuously monitors tray composition and can advise on removing clutter: if certain instruments are definitely not needed for the remainder of the procedure, the cobot 100 can, in some embodiments, reposition or hold those tools aside to free space (this helps prevent “tray congestion” and confusion). Another aspect shown in FIG. 5 is a deviation alert 236 capability. The module 230 is aware of the planned sequence of instrument usage (from the surgical plan or surgeon’s preference list). If the actual usage deviates significantly, for example, an instrument is used out of the expected order, or a backup instrument is opened because a primary one malfunctioned, the system 100 generates an alert or notification. This alert can serve two purposes: (1) to inform the team immediately (e.g., a notification on the user interface 160 or via a subtle audio cue) so they can verify if the deviation was intentional or address any issue (like an instrument problem), and (2) to log the event for postoperative review, since such deviations may be important for quality control or inventory management (for instance, identifying that a certain tool was never used could allow updating the preference card to omit it in the future). The integrated tool and asset management module 230 may also proactively initiate actions: FIG. 5 indicates that if a sterile instrument on the field becomes contaminated (detected perhaps by it falling outside the sterile area or touching an unsterile surface, which the cobot’s vision could recognize), the system 100 can signal for a replacement and even dispatch the cobot (or another connected robotic system) to retrieve a new sterile instrument while alerting staff to remove the contaminated one. In some embodiments, module 230 is tied into the hospital’s central inventory system; thus, FIG. 5 could also represent the cobot updating inventory records or requesting restocking of supplies post-procedure. By closely managing instruments and assets in real time, the module 230 helps prevent delays (no more searching for missing tools), reduce clutter and duplication (only needed instruments are present, others are secured out of the way), and enhance safety (through instrument counts and contamination alerts). This integrated approach ultimately contributes to a smoother surgical workflow and provides a rich dataset of instrument utilization that can be analyzed to optimize tray compositions and preference lists for future cases.

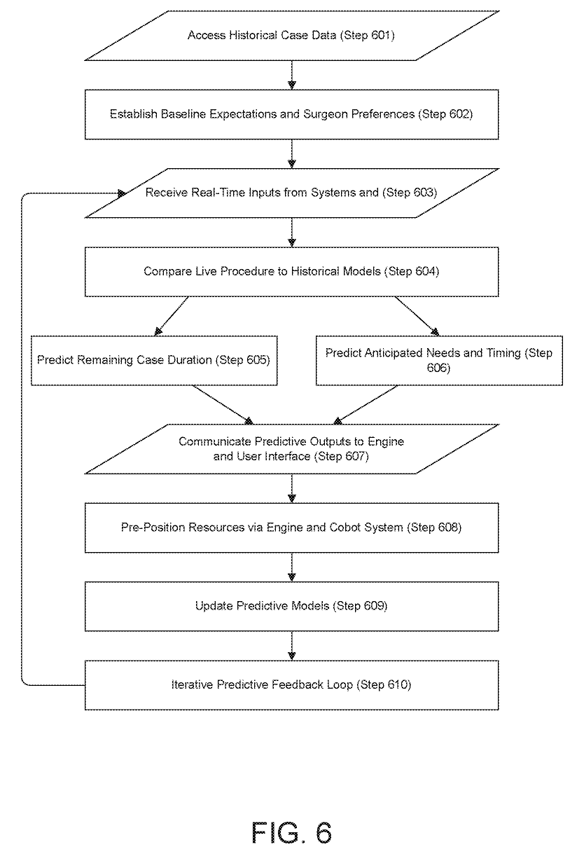

With reference to FIG. 6, a flowchart is shown detailing operation of the predictive workflow optimization engine 240. The predictive workflow optimization engine 240 operates as a continuously updating predictive control process that combines historical surgical knowledge with real-time procedural data to forecast upcoming workflow events and generate optimization outputs.

Access Historical Case Data (Step 601). The predictive workflow optimization engine 240 accesses historical surgical case data 241 relevant to a current surgical procedure, including records of prior similar procedures, historical procedural timelines, outcome metrics, and surgeon-specific practice patterns stored by the cobot system 100 or associated hospital databases.

Establish Baseline Procedural Expectations (Step 602). Using the historical case data 241, the engine 240 establishes baseline expectations for the surgical procedure, including expected workflow phase durations, anticipated instrument usage sequences, and known surgeon preferences 242.

Receive Real-Time Procedural Inputs (Step 603). The engine 240 continuously receives real-time procedural inputs 243 from the ongoing surgery, including a currently recognized workflow phase provided by the workflow monitoring and recognition system 210, instrument usage data provided by the integrated tool and asset management module 230, and indicators of procedural deviations or delays.

Compare Live Procedure to Historical Models (Step 604). The predictive workflow optimization engine 240 applies one or more machine learning models or predictive algorithms to compare the live procedural trajectory to historical patterns and baseline expectations, dynamically adjusting predictive outputs in response to observed deviations from expected progression.

Predict Remaining Case Duration (Step 605). Based on the comparison of real-time inputs and historical models, the engine 240 generates a prediction of remaining surgical case duration 244, representing an updated estimate of time remaining to completion of the surgical procedure.

Predict Anticipated Needs and Timing (Step 606). The engine 240 generates predictions of anticipated procedural needs and timing 245, including predicted upcoming workflow steps, estimated timing for instrument usage, and projected readiness for subsequent phases such as wound closure or staff handoffs.

Communicate Predictive Outputs (Step 607). The predictive outputs generated by the engine 240 are communicated to the adaptive task allocation engine 220 and the user interface 160 to enable proactive cobotic task scheduling and to inform surgical staff of anticipated upcoming events.

Pre-Position Resources (Step 608). Based on the predicted needs and timing, the engine 240 initiates pre-positioning of resources 246 by instructing the adaptive task allocation engine 220 to direct the cobot 100 to fetch, stage, or prepare instruments or equipment in advance, or to generate alerts prompting human personnel to prepare upcoming resources.

Update Predictive Models (Step 609). Following execution of predicted actions and continued observation of procedural outcomes, the engine 240 updates internal predictive model parameters based on observed accuracy of prior predictions and actual procedural progression.

Iterative Predictive Feedback Loop (Step 610). The predictive workflow optimization process repeats continuously throughout the surgical procedure, enabling the engine 240 to refine forecasts in real time and progressively improve prediction accuracy across subsequent surgical cases.

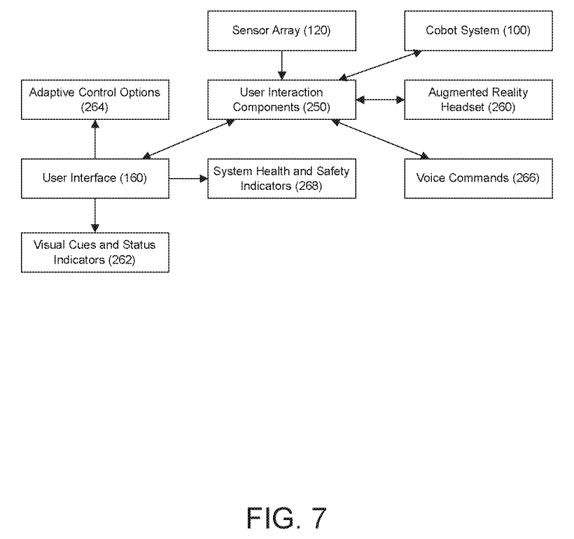

FIG. 7 shows an exemplary user interface layout 160 and related interaction modalities through which the surgical team interacts with the cobot system 100. In the illustrated embodiment, the primary interface 160 is a touchscreen display mounted on the cobot or positioned nearby in the OR. This interface presents a dashboard of visual cues and status indicators 262 to keep the surgical staff informed of the system’s status and insights. For instance, as shown in FIG. 7, the display may include a progress bar or timeline indicating the current detected workflow phase (“Current Phase: Anastomosis, 30% of procedure completed”), with upcoming phases or tasks listed underneath. It can also show icons or color-coded indicators for various subsystems: one section of the UI might display the status of instruments (e.g., a checklist of all instruments with those already used checked off, and any missing tools highlighted in red), another section could show real-time notifications or suggestions from the cobot (for example, “Instrument tray complete, 12/12 instruments accounted for” or “Prep for closing: sutures in 5 minutes”). The visual cues are designed to be intuitive: FIG. 7 illustrates symbols like a small hand icon next to an instrument name to indicate the cobot arm 130 is about to hand that instrument, or a warning triangle if a deviation or safety alert arises. In addition to passive indicators, the user interface 160 supports adaptive control options for the staff. Via the touchscreen, a user can override or modify the cobot’s actions: for example, FIG. 7 shows selectable buttons or menus 264 that allow the surgeon or circulating nurse to request a specific action (“Deliver scalpel now” or “Skip preparing instrument X”), to adjust system settings (like toggling the cobot between autonomous mode and standby), or to acknowledge and dismiss alerts. The system is also equipped with voice interaction capabilities, enabling truly hands-free control when needed. A clinician can speak commands such as “cobot, pause” or “bring the suture kit”, the cobot’s speech recognition module (part of interface components 250) will interpret these in real time. FIG. 7 symbolically shows a microphone icon indicating that voice commands 266 are active; the system may provide voice feedback as well (e.g., a gentle confirmation like “Instrument coming”). Furthermore, the interface extends to augmented reality (AR) headsets and other wearable devices used by the surgical team. In one embodiment depicted in FIG. 7, a surgeon wearing an AR headset 260 sees a heads-up overlay of information in their field of view: for example, the next instrument needed might be highlighted with a glowing outline on the real instrument tray, or a floating text might show “Next step: Place trocar”. The AR interface 260 can also show status indicators (similar to the ones on the touchscreen 160) but superimposed onto the real-world view, ensuring the surgeon doesn’t have to look away from the operative field. Interaction via AR may include gesture controls, for instance, the surgeon could perform a simple hand gesture that the cobot’s cameras 120 recognize as a command (such as pointing at a tray to signal “bring that to me”). The combination of these modalities (touchscreen 160, voice 266, AR 260, and even conventional keyboard/mouse if needed) makes the user interaction system context-aware and robust. The interface adapts its content based on the workflow phase: during critical moments, it might minimize non-essential information and only show alerts if absolutely necessary (to avoid distraction), whereas in calmer phases or during setup, it can present more detailed analytics or options. FIG. 7 also highlights status indicators 268 like system health (battery level of the cobot, network connectivity, etc.) and safety status (whether the cobot’s motion is in restricted mode or normal mode). By providing clear visual/auditory cues and easy ways for staff to give input, the user interface 160 ensures that the cobot 100 remains a transparent and controllable presence in the OR. Surgeons and nurses can trust what the system is doing because they can see its rationale and intervene or redirect it at any time. This professional, surgeon-friendly interface design is key to fostering effective human-cobot collaboration in the high-stakes surgical setting.

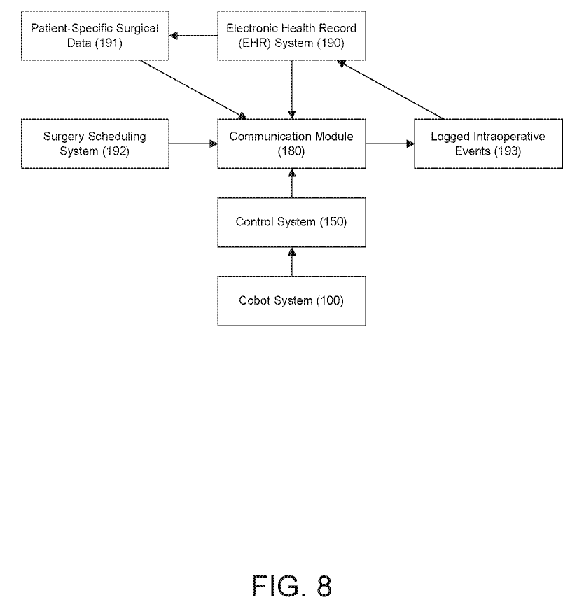

Moving on to FIG. 8, this communication diagram illustrates the cobot system’s integration with external hospital information systems, notably the electronic health record (EHR) system 190 and the surgery scheduling system 192. As shown in FIG. 8, the cobot’s communication module 180 (which may be a network interface supporting hospital Ethernet/Wi-Fi or a secure wireless link) enables bidirectional data exchange between the cobot control system 150 and the hospital’s digital infrastructure. In practice, before a procedure begins (e.g., during the preoperative phase), the control system 150 queries the scheduling system 192 and EHR 190 to retrieve the surgical plan and patient-specific data 191. This can include the scheduled procedure type, the patient’s ID and relevant medical history or special considerations (allergies, implant records, etc.), the surgeon’s name and known preferences (often stored as part of a surgeon’s profile in the system), and a list of required instruments or implants for that case (the preference card or pick list). Upon retrieval, the cobot 100 uses this information to configure its subsystems: for example, the integrated tool management module 230 cross-references the expected instrument list to ensure those instruments are present and tracked, and the predictive engine 240 tailors its models using surgeon-specific data. FIG. 8 indicates this by an arrow from EHR 190 to the cobot system 100, representing the download of case data. During the surgery, the communication integration continues to play a role. The cobot’s control system 150 can log intraoperative events 193 and push them to the patient’s record or to a centralized case log in real time. Key events such as “incision made”, “clamp applied”, “procedure step X completed at 10:32 AM” can be automatically time-stamped and sent through module 180 to the EHR system 190 (or an operating room management system) as part of the permanent record. FIG. 8 shows arrows from the cobot 100 back to the hospital systems, denoting this event logging and data upload. In some embodiments, the system also transmits continuous data streams or summaries, for instance, uploading vitals, surgical robotic utilization data and significant occurrences to a monitoring dashboard for remote supervisors or for AI analytics. The integration depicted in FIG. 8 further allows the cobot 100 to stay updated if there are changes: for example, if a last-minute update to the surgical plan is entered in the EHR (such as an added procedure or a change in implant type), the control system 150 will receive that update via the communication module 180 and adapt the workflow accordingly (perhaps by fetching a different instrument set or updating the predictive engine’s expectations). Moreover, linking with the scheduling system 192 means the cobot knows about downstream cases and overall OR timing, which could influence its behavior if, say, the current case is running long and the next patient is waiting (the system might notify staff or speed up certain non-critical steps if safe). The communication module 180 employs secure protocols for all these exchanges, ensuring compliance with patient privacy and data security regulations (for example, using encryption and authentication as required by hospital IT policies). By logging intraoperative events automatically, the cobot 100 relieves some of the documentation burden on clinicians, allowing them to focus on patient care. After the procedure, the comprehensive log (including the timeline of events, instrument usage statistics, etc., compiled via this integration) is available for postoperative review and audit. Surgeons can review this data to gain insights into the procedure (e.g., to evaluate efficiency or for training purposes), and hospital administrators can use it for quality improvement (for instance, analyzing time stamps to identify workflow delays). In summary, FIG. 8 demonstrates that the cobot system 100 is not an isolated robot, but rather a connected part of the hospital’s ecosystem, pulling in schedule and patient data to inform its actions and pushing out detailed operative records to enhance documentation and subsequent analysis.

Lastly, FIG. 9 depicts the safety and compliance mechanisms incorporated into the cobot system 100, ensuring that all the aforementioned adaptive functions operate without compromising the sterile environment or staff safety. In FIG. 9, a number of safety features are illustrated schematically. One key feature is sterile-zone detection and enforcement. The cobot 100, through its sensor array 120 and control algorithms, is constantly aware of the location of the sterile field 272 and other no-go zones in the OR. For example, depth sensors and cameras (possibly indicated in FIG. 9 by coverage cones around the cobot) map out the region of the patient and instrument table that constitutes the sterile field. The control system 150 creates a virtual boundary around this area (shown as a boundary line in FIG. 9) and will automatically constrain the motion of the cobot such that no part of the base 110 or arms 130 intrudes into the sterile field unless explicitly allowed. In practice, this means even if the cobot is tasked to deliver an instrument, it will stop at the edge of the sterile zone and extend the instrument only into a hand-off area, or it may place items on a sterile transfer tray, ensuring the integrity of sterility. If the surgical team wants the cobot to perform an action within the sterile field (e.g., hold a camera boom that is in the field), the cobot can be draped sterile or otherwise authorized, but such actions are under controlled conditions. FIG. 9 also illustrates the cobot’s collision avoidance protocols. The sensor array 120 detects the presence of any personnel or obstacles around the cobot, for instance, if a person comes within a defined safety radius of the moving arm 130 or if a cart is suddenly pushed into the cobot’s path, the system will immediately adjust. The control system 150 runs an obstacle avoidance algorithm that can dynamically stop movement or alter the cobot’s path to avoid contact. In FIG. 9, this is symbolized by an array of proximity sensors around the cobot and arrows showing a safe rerouting path around a person 274. Additionally, the cobot 100 is equipped with multiple layers of override controls and failsafes. As shown in FIG. 9, an emergency stop control 276 is accessible to staff, this could be a physical red button on the cobot’s body and/or a soft emergency stop button on the touchscreen interface 160. If at any time a staff member presses this emergency stop, the cobot 100 will immediately halt all motion and power down to a safe state, regardless of what task it was performing. Similarly, the system’s software includes watchdog timers and fault detection; if any subsystem of the cobot deviates from expected behavior (for example, a joint motor not responding or a sensor fault that could impair situational awareness), the control system 150 will default to a safe mode (pausing the cobot and alerting the team). The override and escalation protocols built into the system ensure that human personnel can always assume direct control: for instance, FIG. 9 shows a scenario where if an unusual situation (a “workflow anomaly” or an unmodeled emergency) is detected by the system, it will notify the team (via an alarm or UI message) and await human direction, effectively handing over decision-making until things stabilize. The cobot 100 is also designed to be mechanically compliant, its arms 130 are force-limited so that if they do come into unintended contact with a person, they either yield or apply only a gentle force below a safe threshold. In FIG. 9, this is indicated by a force-feedback sensor symbol on the arm. The system additionally tracks its own speed and route to avoid any sudden movements that could startle or impact staff. All motions near people are smooth and deliberate, and the cobot can audibly announce its intentions (e.g., “moving to your left”) if programmed to do so, to further mitigate surprise. In summary, FIG. 9 highlights that the safety, redundancy, and workflow compliance features are integral to the cobot’s operation. The system 100 continuously monitors its environment and its own actions to ensure it does not interfere with critical surgical activities or sterile technique. It provides immediate manual override options and graceful degradation in case of anomalies. These measures collectively allow the cobot to be a reliable assistant that augments the surgical workflow without ever becoming a hazard or hindrance. In the event of any conflict between automated actions and surgical team actions, the system is designed to yield to the humans, pausing or adapting as directed. Thus, the adaptive perioperative cobot system not only optimizes surgical workflow through the intelligent features described above, but does so with a safety-first design that aligns with the stringent requirements of the operating room.

While specific embodiments have been described to enable practice of the disclosed subject matter, they are illustrative and not limiting. Variations, modifications, substitutions, and combinations of the features described can be made by those skilled in the art without departing from the scope of this disclosure. Features described with respect to one embodiment may be used with, or substituted for, features of another. References to “RFID” should be interpreted broadly to include similar technologies such as Bluetooth LE, Zigbee and RuBee. Unless expressly stated otherwise, the terms “comprising,” “including,” and “having” are used in an open, non‑limiting sense; the singular forms “a,” “an,” and “the” include the plural forms; and stated ranges include their endpoints and all subranges. Unless the context clearly indicates otherwise, “and/or” means one or more of the listed items (e.g., “A and/or B” encompasses A alone, B alone, or both A and B); “or” is inclusive (i.e., “A or B” means A, B, or both); and “and” is conjunctive. Headings and ordering are for convenience and do not limit the description. The scope of the claimed subject matter is defined solely by the claims that follow and their legal equivalents.

What is claimed is:

1. A perioperative cobotic system for surgical workflow optimization, comprising:
a cobot having a base, at least one articulated robotic arm, and a set of surgical-environment sensors, the cobot configured to perform one or more perioperative tasks;
a workflow monitoring and recognition system comprising a machine learning module operatively coupled to the sensors and configured to classify workflow phases of a surgical procedure and monitor surgical team behavior based on data from the sensors;
an adaptive task allocation engine configured to dynamically assign tasks to the cobot based on the classified workflow phase and the actions of the surgical team;
an integrated tool and asset management module configured to track real-time usage of surgical instruments and other surgical assets;
a predictive workflow optimization engine configured to utilize historical surgical data and real-time procedure data to anticipate upcoming workflow needs and optimize the cobot’s task schedule;
a context-aware user interaction system configured to communicate with surgical staff and to receive user inputs via voice commands, gestures, or an augmented reality interface;
an electronic health record integration module configured to retrieve patient-specific surgical information and procedural plans from an EHR system and to log intraoperative events and timestamps to the EHR system; and
a safety and compliance module configured to monitor sterile field boundaries and enable manual override of the cobot’s operations.

2. The system of claim 1, wherein the cobot comprises a mobile base configured for autonomous navigation within an operating room, the cobot performing real-time path planning, obstacle avoidance, and maintaining compliance with sterile-field boundaries during movement.

3. The system of claim 1, wherein the set of surgical-environment sensors includes at least one optical imaging sensor, at least one audio sensor, and at least one motion tracking sensor, and wherein the machine learning module of the workflow monitoring and recognition system is trained to identify discrete workflow phases including an incision phase, a retraction phase, and a closure phase based on data from the set of surgical-environment sensors.

4. The system of claim 1, wherein the adaptive task allocation engine is configured to assign the cobot to perform one or more support tasks, including delivering surgical instruments, adjusting operating room equipment, or performing environmental adjustments, based on the current workflow phase and the actions of the surgical team.

5. The system of claim 1, wherein the adaptive task allocation engine is further configured to reprioritize pending cobot tasks in response to a detected workflow deviation, delay, or change in surgical personnel, thereby adjusting the cobot’s actions in real time to maintain workflow continuity.

6. The system of claim 1, wherein the integrated tool and asset management module is configured to issue an alert or initiate a reprocessing procedure when an instrument’s usage deviates from a predetermined surgical plan or when a sterilization status threshold for an instrument is violated.

7. The system of claim 1, wherein the predictive workflow optimization engine is configured to analyze historical surgical case data, surgeon preference data, and current procedure data to predict upcoming workflow steps and an expected duration of the surgical procedure, and to update predictive models based on outcomes of completed procedures to improve future workflow predictions.

8. The system of claim 1, wherein the context-aware user interaction system includes a voice recognition interface and an augmented reality display, the system being configured to accept spoken or gesture-based commands from the surgical staff and to provide real-time visual cues or status updates to the surgical team during the procedure.

9. The system of claim 1, wherein the electronic health record integration module is configured to automatically retrieve a scheduled surgical procedure plan and patient-specific requirements from the EHR system before the surgery, and to document intraoperative events, including workflow phase transitions and instrument usage, in the patient’s electronic health record in real time.

10. The system of claim 1, wherein the safety and compliance module comprises a sterile-field detection subsystem that prevents the cobot from breaching a sterile field boundary, and a manual override control configured to immediately halt or modify the cobot’s actions upon activation by a user.

11. The system of claim 1, wherein the context-aware user interaction system is configured to present predictive cues or recommendations generated by the predictive workflow optimization engine to the surgical team via an augmented reality display, thereby providing the surgical team with information about anticipated next steps in the procedure.

12. A method for perioperative surgical workflow monitoring and data logging using a collaborative robotic system, the method comprising:
collecting, by a cobot equipped with optical, audio, and motion tracking sensors, multimodal data from an operating room during a surgical procedure;
analyzing, in real time, the collected data using a machine learning algorithm to determine a current workflow phase of the surgical procedure and to detect actions of a surgical team including usage of surgical instruments;
recording, in a structured log, a plurality of entries each comprising a timestamp and an indication of a detected workflow phase or event along with associated instrument usage information; and
storing the structured log of the surgical procedure in an electronic health record system.

13. The method of claim 12, wherein collecting the multimodal data comprises capturing video of the surgical field via the optical sensor, capturing ambient audio via the audio sensor, and tracking movements of surgical personnel or equipment via the motion tracking sensors.

14. The method of claim 12, wherein collecting the multimodal data further comprises detecting usage of surgical instruments by identifying instrument identifiers or movements using at least one of RFID technology or computer vision, and including corresponding instrument usage data in the structured log.

15. The method of claim 12, wherein analyzing the collected data using the machine learning algorithm includes applying a trained model that recognizes predefined workflow phases of the surgical procedure, including an incision phase, a retraction phase, and a closure phase, based on patterns in the optical, audio, and motion data.

16. The method of claim 12, wherein storing the structured log in the electronic health record system comprises automatically populating an intraoperative record in a patient’s electronic health record with the timestamped workflow phase entries and associated instrument usage data.

17. The method of claim 12, further comprising using the structured log data from the surgical procedure to update the machine learning algorithm, thereby improving the accuracy of workflow phase recognition for subsequent procedures.

18. A surgical workflow optimization system, comprising:
a cobot including at least one robotic arm and a set of sensors configured to monitor an ongoing surgical procedure;
an EHR integration module configured to retrieve patient-specific surgical data and procedural plans from an EHR system; and
a predictive workflow optimization engine operatively coupled to the sensors and to the EHR integration module, the predictive workflow optimization engine being configured to analyze data from the sensors in conjunction with historical surgical data and the retrieved patient-specific surgical data to predict one or more upcoming workflow events during the surgical procedure, and to automatically adjust a task-execution schedule of the cobot in real time based on the predicted upcoming workflow events.

19. The system of claim 18, wherein the electronic health record integration module retrieves surgeon-specific preference data and patient health information, and the predictive workflow optimization engine adjusts the predicted upcoming workflow events based on the retrieved surgeon preferences and patient-specific risk factors.

20. The system of claim 18, wherein the predictive workflow optimization engine is configured to estimate an expected remaining duration of the surgical procedure and to generate a notification or recommendation for operating room staff, including coordination of personnel breaks or handoffs, based on the predicted progression of the procedure.
